(19) United States
(12) Patent Application Publication
(10) Pub. No.: US 2005/0143323 A1
(43) Pub. Date: Jun. 30, 2005

(54) Isoflavone therapy for treating urinary incontinence

(76) Inventor: E. C. Henley, Athens, GA (US)

Correspondence Address: SOLAE, LLC;LEGAL DEPARTMENT, BUILDING 3C, P.O. BOX 88940, ST. LOUIS, MO, 63188, US

(21) Appl. No.: 10/748,492

(22) Filed: Dec. 30, 2003

(51) Int. Cl. A61K 31/7048; A61K 31/353

(52) U.S. Cl. 514/27; 514/456

(57) ABSTRACT

A method for preventing or inhibiting urge or stress urinary incontinence while reducing or eliminating undesirable side effects associated with conventional treatments involves administering to an human in need of treatment an effective amount of an isoflavone.

### FIELD OF THE INVENTION

This invention relates to the treatment of disease, and more particularly to the treatment of urinary incontinence.

### BACKGROUND OF THE INVENTION

Urinary incontinence is a disorder characterized by uncontrollable leakage of urine. It is embarrassing, uncomfortable, and can directly affect a person's health and quality of life. It has been estimated that urinary incontinence affects over 10,000,000 Americans each year, and costs related to incontinence are over $4,000,000,000 a year.

Incontinence is classified into four basic categories: transient incontinence, urge incontinence, stress incontinence and overflow incontinence. Transient incontinence is due to factors outside the urinary tract such as drugs or social and mobility issues that affect the ability to move one self into the bathroom. It may be related to the level of consciousness and perception that a person has of their surroundings. For example, certain drugs, such as sedatives, may cause an elderly patient to become confused and urinate in bed. Urge incontinence is described by patients as the urge to urinate but being unable to hold the urine in time to get to a bathroom. It is characterized as an uncontrolled spasm of the bladder muscle resulting in its contraction and emptying of urine. These uncontrolled spasms may occur without the patient being aware. Urge incontinence can be caused by a variety of diseases such as diabetes, strokes, Parkinson's disease, as well as simple urinary tract infections.

Stress incontinence is associated with leakage of urine when coughing, sneezing, or performing a strenuous activity. These stress events may cause an increase in pressure from outside the bladder. Normally, the sphincter and support structures at the base of the bladder keep urine from leaking out. However, if any of the structures are damaged or weakened from events such as surgery, childbirth or other diseases, stress incontinence may occur.

Overflow incontinence occurs when the bladder is filled to capacity and spills out. This can occur in situations when the bladder muscle is weak and unable to contract or when there is a blockage causing the bladder to not empty properly until it is too full. Such blockages typically occur in men with large obstructing prostate glands. Bladder muscle weakness can often occur in patients whose nerves to the bladder are injured after surgery, from diabetes or other disease of the nervous system.

Treatments for incontinence range from simple behavioral training exercises to drugs and surgery. Drugs which have been used for the treatment of urinary incontinence include antibiotics, anticholinergic drugs, and estrogen. Most drugs currently used for treating urinary incontinence exhibit some undesirable side effects. For example, anticholinergic drugs may cause abdominal cramps or discomfort, nausea, belching, diarrhea, bronchial constriction, asthmatic attacks, malaise, headaches, dizziness, lightheadedness, and other undesirable effects. Estrogens may increase the risk of endometrial carcinoma, may cause elevated blood pressure, and may increase the risk of gallbladder disease, thromboembolic disease, and other undesirable effects. Antibiotics commonly used for treating urinary incontinence can cause nausea, vomiting, abnormal cramps/pain, diarrhea, headache, dizziness, rash, pruritus, edema, renal function impairment, and other undesirable effects.

Thus, a need exists for treating urinary incontinence with chemical agents which exhibit fewer, and less intense, undesirable effects. More desirably, the treatment would involve the use of a naturally occurring constituent of an agricultural product.

### SUMMARY OF THE INVENTION

In one aspect, the invention provides a method for preventing or inhibiting stress or urge urinary incontinence while minimizing exposure to undesirable effects. The method involves administering to a human a composition containing at least one isoflavone selected from the group consisting of genistein, daidzein, glycitein, biochanin A, formononetin, their naturally occurring glycosides, their naturally occurring glycoside conjugates, or mixture thereof in an amount which is effective to prevent or inhibit stress or urge urinary incontinence in the human.

### DESCRIPTION OF THE PREFERRED EMBODIMENTS

As used herein “Mal” represent malonyl and “Ac” represents acetyl. The word “isoflavone” as used herein may mean a single isoflavone or plural isoflavones, depending on the context.

The present invention resides in the discovery that a select group of isoflavones—genistein, genistin, 6″-O-Mal genistin, 6″-O-Ac genistin, daidzein, daidzin, 6″-O-Mal daidzin, 6″-O-Ac daidzin, glycitein, glycitin, 6″-O-Mal glycitin, biochanin A, and formononetin, shown in Formulas 1 and 2 below—are effective in preventing or reducing stress and urge urinary incontinence.

Formula 1

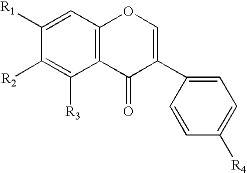

| Compound | R₁ | R₂ | R₃ | R₄ |
|---|---|---|---|---|
| Genistein | OH | H | OH | OH |
| Daidzein | OH | H | H | OH |
| Glycitein | OH | OCH₃ | H | OH |
| Biochanin A | OH | H | OH | OCH₃ |
| Formononetin | OH | H | H | OCH₃ |

Formula 2

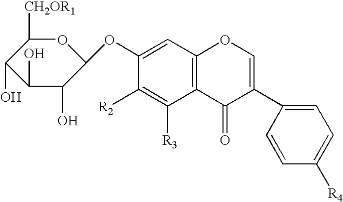

| Compound | R₁ | R₂ | R₃ | R₄ |
|---|---|---|---|---|
| Genistin | H | H | OH | OH |
| 6′-OMal genistin | COCH₂CO₂H | H | OH | OH |
| 6′-OAc genistin | COCH₃ | H | OH | OH |
| Daidzin | H | H | H | OH |
| 6′-OMal daidzin | COCH₂CO₂H | H | H | OH |
| 6′-OAc daidzin | COCH₃ | H | H | OH |
| Glycitin | H | OCH₃ | H | OH |
| 6′-OMal glycitin | COCH₃ | OCH₃ | H | OH |

The isoflavones of Formulas 1 and 2 are selective estrogen receptor modulators which exhibit estrogenic activity in certain tissues, including human male and female smooth muscle urethra tissues, while exerting anti-estrogenic activity in other tissues, particularly human female breast and uterine tissues. Estrogen is known to increase the density of adrenergic receptors and the adrenergically mediated contractile responses of urethral smooth muscle tissues, and is also known to innervate the striated urethral sphincter and pelvic floor muscles. One mechanism by which the isoflavones of Formulas 1 and 2 inhibit or prevent stress or urge urinary incontinence may be similar to estrogen-mediated increased contractile response in the urethral smooth muscle tissues, whereby the increased contractile response of the urethral smooth muscle tissues prevents uncontrolled micturation in response to a bladder spasm or from stress on the bladder. Another mechanism by which the isoflavones of Formulas 1 and 2 inhibit or prevent stress or urge urinary incontinence may be similar to estrogen-mediated innervation of the striated urethral and pelvic floor muscles, whereby the innervated striated urethral and pelvic floor muscles also prevent uncontrolled micturation in response to a bladder spasm or from stress on the bladder.

The isoflavone compounds of Formulas 1 and 2 are naturally occurring substances which may be found in plants such as legumes, clover, and the root of the kudzu vine (pueraria root). Common legume sources of these isoflavone compounds include soy beans, chick peas, and various other types of beans and peas. Clover sources of these isoflavone compounds include red clover and subterranean clover. Soy beans are a particularly preferred source of the isoflavone compounds (except biochanin A and formononetin which are not present in soy).

The isoflavone compounds of Formulas 1 and 2 may be isolated from the plant sources in which they naturally occur, and several of the isoflavone compounds of Formulas 1 and 2 may be synthetically prepared by processes known in the art. For example, daidzein may be isolated from red clover as disclosed by Wong (J. Sci. Food Agr., Vol. 13, p. 304 (1962)) or may be isolated from the mold Micromonospora halophytica as provided by Ganguly and Sarre (Chem. & Ind. (London), p. 201 (1970)), both references of which are incorporated by reference herein. Daidzein may be synthetically prepared by the methods provided by Baker et al (J. Chem. Soc., p. 274 (1933)), Wesley et al. (Ber. Vol. 66, p. 685 (1933)), Mahal et al. (J. Chem. Soc., p. 1769 (1934)), Baker et al. (J. Chem. Soc., p. 1852 (1953)), or Farkas (Ber. Vol. 90, p. 2940 (1957)), each reference of which is incorporated herein by reference. Daidzin may be synthetically prepared by the method of Farkas et al. (Ber., Vol. 92, p. 819 (1959)), incorporated herein by reference. The daidzein isoflavone glucoside conjugates 6′-OMal daidzin and 6′-OAc daidzin can be prepared by a conventional saponification of daidzin with a malonyl or an acetyl anhydride, respectively.

Genistein may be synthetically prepared by the methods provided by Baker et al (J. Chem. Soc., p. 3115 (1928)); Narasimhachari et al. (J. Sci. Ind. Res., Vol. 12, p. 287 (1953)); Yoder et al., (Proc. Iowa Acad. Sci., Vol. 61, p. 271 (1954); and Zemplen et al. (Acta. Chim. Acad. Sci. Hung., Vol. 19, p. 277 (1959)), each reference of which is incorporated herein by reference. Genistin may be synthetically prepared by the method of Zemplen et al. (Ber., Vol 76B, p. 1110 (1943)), incorporated herein by reference. The isoflavone glucoside conjugates 6′-OMal genistin and 6′-OAc genistin can be prepared by a conventional saponification of genistin with a malonyl or an acetyl anhydride, respectively.

Biochanin A can be synthetically prepared by the method provided by Baker et al. (Nature 169:706 (1952)), incorporated herein by reference. Biochanin A can also be separated from red clover by the method provided by Pope et al. (Chem. & Ind. (London) p. 1092 (1953)), incorporated herein by reference. Formononetin can be synthetically prepared by the methods disclosed by Wessely et al. (Ber. 66:685 (1933)) and Kagel et al. (Tetrahedron Letters, p. 593 (1962)), both references of which are incorporated herein by reference. Formononetin can be isolated from soybean meal by the method of Walz (Ann. 489:118 (1931)) or can be isolated from clover species by the method of Bradbury et al. (J. Chem. Soc. p. 3447 (1951)), both references of which are incorporated herein by reference.

A preferred method of isolating the isoflavone compounds of Formulas 1 and 2 from plant materials in which they naturally occur is to extract the plant materials with an alcohol, preferably methanol or ethanol, or an aqueous solution, preferably an aqueous alkaline solution, to remove the isoflavones from the plant material. It is preferred to comminute the plant material before extracting the isoflavone compounds to maximize recovery of the isoflavone compounds from the plant material. The isoflavone compounds can be isolated from the extract by conventional separation procedures such as reverse phase high performance liquid chromatography (“HPLC”).

In a preferred embodiment, the isoflavone compounds genistein, genistin, 6′-OMal genistin, 6′-OAc genistin, daidzein, daidzin, 6′-OMal daidzin, 6′-OAc daidzin, glycitein, glycitin, and 6′-OMal glycitin are isolated from a soy material, preferably a commercially available soy material. Soy materials from which the isoflavone compounds can be isolated include: soy beans, dehulled soy beans, soy meal, soy flour, soy grits, soy flakes (full fat and defatted), soy cotyldeons, soy hypocotyls, soy molasses, soy protein concentrate, soy whey, soy whey protein, and soy protein isolate. In one embodiment, the isoflavones are extracted from soy beans, dehulled soy beans, soy cotyledons, soy hypocotyls, soy meal, soy flour, soy grits, soy flakes, soy protein concentrate, soy whey protein, or soy protein isolate, preferably soy meal, soy flour, soy grits, or soy flakes, with a low molecular weight organic extractant, preferably an alcohol, ethyl acetate, or acetone, and most preferably aqueous ethyl alcohol or aqueous methyl alcohol. In a preferred embodiment, the isoflavones are separated into the low molecular weight organic extractant by refluxing the soy material in the extractant for a period of from about 1 to 4 hours. Most preferably the extractant has a pH of about the isoelectric point of soy protein (about pH 4 to pH 5) to minimize the amount of soy protein extracted by the extractant.

The extractant containing the isoflavones is separated from the insoluble soy materials to form an isoflavone rich extract. If desired, an isoflavone rich material may be recovered by concentrating the extract to remove the solvent thereby producing a solid isoflavone containing material.

In a more preferred embodiment the isoflavone compounds are further purified from other soy materials soluble in the isoflavone rich extract by contacting the extract with a material which adsorbs the isoflavones in the extract, and eluting the adsorbed isoflavones out of the adsorbent material with a solvent which causes the isoflavones to be differentially eluted from the adsorbent material.

In a preferred embodiment, the isoflavones are separated from impurities in the isoflavone rich extract by a conventional reverse phase HPLC separation. After extraction of the isoflavones from the soy material and separation of the isoflavone rich extract from the insoluble soy materials, the extract is filtered to remove insoluble materials that could plug an HPLC column. An HPLC column is prepared by packing a conventional commercially available HPLC column with a particulate adsorbent material which will releasably bind the isoflavones and impurities in the extract in a compound specific manner. The adsorbent material may be any reverse phase HPLC packing material, however, a preferred packing material may be chosen by the criteria of load capacity, separation effectiveness, and cost. One such preferred packing material is Kromasil C18 16 μm 100 Å beads available from Eka Nobel, Nobel Industries, Sweden.

The filtered isoflavone rich extract is passed through the packed HPLC column until all the binding sites of the column are fully saturated with isoflavones, which is detected by the appearance of isoflavones in the effluent from the column. The HPLC column may then be eluted with a solvent to effect the separation. In a preferred embodiment, the eluent is a polar solvent such as ethanol, methanol, ethyl acetate, or acetonitrile, and preferably is an aqueous alcohol having an alcohol content of between about 30% and about 90%, most preferably about 50%, and most preferably the alcohol is ethanol.

The isoflavone compounds and impurities are separately collected from the column effluent. The isoflavone fractions of the eluent may be identified from other eluent fractions in accordance with conventional HPLC and analytical chemistry techniques.

The isoflavone fractions of the eluent may be collected from the column, and the volatile content of the solvent (e.g. alcohol) can be removed by evaporation. The isoflavone compounds can be recovered directly if the all of the solvent is removed by evaporation, or may be recovered by chilling the remaining solvent (e.g. water) to crystallize the isoflavones, and centrifuging or filtering the crystallized isoflavones from the remaining solvent.

In a particularly preferred embodiment the soy isoflavone glucoside conjugates—6′-OMal genistin, 6′-OAc genistin, 6′-OMal daidzin, 6′-OAc daidzin, and 6′-OMal glycitin—and the soy isoflavone glucosides—genistin, daidzin, and glycitin—are converted to their respective aglucone isoflavone forms—genistein, daidzein, and glycitein. The conversion of the isoflavone conjugates and isoflavone glucosides to the aglucone isoflavones can be effected in the soy substrate from which the isoflavones are to be extracted prior to the extraction, or may be effected in the isoflavone rich extract after separation of the extract from the insoluble soy materials. The aglucone isoflavone compounds are believed to be particularly active in preventing or reducing stress or urge urinary incontinence since the aglucone isoflavones appear to be more bioavailable than the isoflavone glucosides and the isoflavone glucoside conjugates. Furthermore, the aglucone isoflavones are more easily separated from an extract containing water than their respective conjugate and glucoside forms since the aglucones are less water soluble.

The isoflavone conjugates 6″-O-Mal genistin, 6″-O-Ac genistin, 6″-O-Mal daidzin, 6″-O-Ac daidzin, and 6″-O-Mal glycitin can be converted to their respective glucosides genistin, daidzin, and glycitin by forming an aqueous alkaline solution of the soy substrate containing the isoflavones having a pH of about 6 to about 13, preferably about pH 9 to about pH 11, and treating the aqueous alkaline solution at a temperature of about 2° C. to about 121° C., preferably about 25° C. to about 75° C., for a period of time sufficient to effect the conversion, preferably about 30 minutes to about 5 hours, more preferably about 30 minutes to about 1.5 hours. The isoflavone glucosides genistin, daidzin, and glycitin can be converted to their respective aglucone forms genistein, daidzein, and glycitein by contacting the isoflavone glucosides with an enzyme capable of cleaving a 1,4-β-glucoside bond—preferably a commercially available beta-glucosidase enzyme, an alpha- or beta-galactosidase enzyme, a pectinase enzyme, a lactase enzyme, or a gluco-amylase enzyme—at a pH at which the enzyme is active, typically from about pH 3 to about pH 9, and at a temperature of about 25° C. to about 75° C., more preferably about 45° C. to about 65° C., for a period of time sufficient to effect the conversion, typically about 1 hour to about 24 hours, and preferably about 1 hour to about 3 hours.

The aglucone isoflavones can be separated from the soy substrate using conventional separation procedures. For example, the aglucone isoflavones may be extracted from the soy substrate with a low molecular weight alcohol. The aglucone isoflavones may be separated from the extract by conventional recrystallization processes, or by HPLC. In a particularly preferred embodiment, an isoflavone composition isolated from a soy substrate for formulation into a pharmaceutical composition or a dietary composition for use in the method of the present invention includes at least 40% genistein, at least 15% daidzein, and at least 1% glycitein. In another particularly preferred embodiment of the invention, an isoflavone composition isolated from a soy substrate for formulation into a pharmaceutical composition or a dietary composition for use in the method of the present invention contains at least 85% genistein, at least 5% daidzein, and at least 0.5% glycitein.

Several of the isoflavone compounds of Formula 1 and Formula 2 are commercially available, and may be purchased for formulation into pharmaceutical, dietary supplement, or dietary compositions useful in the method of the present invention. For example, genistein, daidzein, and glycitein are commercially available and may be purchased, for example, from Indofine Chemical Company Inc., P.O. Box 473, Somerville, N.J. 08876, and biochanin A is available from Aldrich Chemical Company, Inc., 940 West Saint Paul Avenue, Milwaukee, Wis. 53233.

To prevent or inhibit stress or urge urinary incontinence in a human, a composition containing at least one isoflavone, preferably at least two isoflavones, selected from the isoflavone compounds discussed above—genistein, daidzein, glycitein, biochanin A, and formononetin; their naturally occurring glucosides genistin, daidzin, and glycitin; and their naturally occurring glucoside conjugates 6″-O-malonyl genistin, 6″-O-acetyl genistin, 6″-O-malonyl daidzin, 6″-O-acetyl daidzin, and 6″-O-malonyl glycitin—is administered to a human, preferably daily or on some other regular basis, in an amount effective to prevent or inhibit urinary incontinence. Preferably the composition containing the isoflavone(s) is a pharmaceutical composition for administration as a drug, a dietary supplement composition for administration as a dietary supplement, or a dietary composition for administration as a food.

A pharmaceutical composition or a dietary supplement composition for use in accordance with the method of the present invention is a composition containing at least one, but preferably at least two, of the isoflavone compounds of Formulas 1 and/or 2 and an excipient. Pharmaceutical and dietary supplement compositions incorporating the isoflavone compounds of Formulas 1 and/or 2 can be prepared by procedures known in the art. For example, the isoflavone compounds can be formulated into tablets, capsules, powders, suspensions, solutions for parenteral administration including intravenous, intramuscular, and subcutaneous administration, and into solutions for application onto patches for transdermal application with common and conventional carriers, binders, diluents, and excipients.

In a preferred embodiment, a pharmaceutical or dietary supplement composition for use in the methods of the present invention includes an isoflavone material containing at least 40% genistein, at least 15% daidzein, and at least 1% glycitein. In another preferred embodiment, a pharmaceutical or dietary supplement composition includes an isoflavone material containing at least 85% genistein, at least 5% daidzein, and at least 0.5% glycitein.

Inert pharmaceutically acceptable carriers useful to form pharmaceutical and dietary supplement compositions in accordance with the present invention include starch, mannitol, calcium sulfate, dicalcium phosphate, magnesium stearate, silicic derivatives, and/or sugars such as sucrose, lactose, and glucose. Binding agents include carboxymethyl cellulose and other cellulose derivatives, gelatin, natural and synthetic gums including alginates such as sodium alginate, polyethylene glycol, waxes and the like. Diluents useful in the invention include a suitable oil, saline, sugar solutions such as aqueous dextrose or aqueous glucose, and glycols such as polyethylene or polypropylene glycol. Other excipients include lubricants such as sodium oleate, sodium acetate, sodium stearate, sodium chloride, sodium benzoate, talc, and magnesium stearate, and the like; disintegrating agents including agar, calcium carbonate, sodium bicarbonate, starch, xanthan gum, and the like; and adsorptive carriers such as bentonite and kaolin. Coloring and flavoring agents may also be added to the pharmaceutical formulations.

A dietary composition for use in accordance with the method of the present invention is a food ingredient or a food containing at least one, but preferably at least two, of the isoflavone compounds of Formulas 1 and/or 2. Dietary compositions incorporating the isoflavone compounds of Formulas 1 and/or 2 can be prepared by adding the isoflavone compounds to a food or a food ingredient in the process of preparing a food, independent of the source from which the isoflavone compounds are derived. The foods to which the isoflavone compounds may be added include almost all foods. For example, the isoflavone compounds can be added to foods including, but not limited to, meats such as ground meats, emulsified meats, marinated meats, and meats injected with the isoflavone compounds; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-dairy frozen desserts; yogurts; soups; puddings; bakery products; salad dressings; dips and spreads such as mayonnaise and chip dips; and extruded snack products.

The isoflavone compounds are added to the food in an amount selected to deliver a desired dose of the compounds to the consumer of the food. In a preferred embodiment, an isoflavone compound added to a food for use as a dietary composition in accordance with the methods of the present invention contains at least 40% genistein, at least 15% daidzein, and at least 1% glycitein. In another preferred embodiment, an isoflavone compound added to a food contains at least 85% genistein, at least 5% daidzein, and at least 0.5% glycitein.

In a particularly preferred embodiment of the invention, the isoflavone compounds are administered to prevent or inhibit stress or urge urinary incontinence in a dietary composition containing a soy protein material. Soy protein materials, as indicated above, may contain significant amounts of the isoflavone compounds of Formulas 1 and 2, except biochanin A and formononetin and their naturally occurring glycosides and glycoside conjugates, which are not present in significant amounts in soy. The soy protein material may be used as a food ingredient in other foods, or may be administered by itself, for example, as a nutritional supplement.

A soy protein material for use in accordance with the method of the present invention is a whole soybean seed, or soy protein derivatives that can be formed from whole soybeans. Soy protein derivatives of whole soybeans include fat-containing or defatted: soy protein isolates, soy protein concentrates, soy flours, and soy meals which are formed in accordance with conventional methods for forming such materials. Soy protein derivatives of whole soybeans also include peptide materials which are formed by hydrolyzing soy protein containing materials in accordance with conventional methods for hydrolyzing soy protein materials, such as enzymatic or acid hydrolysis.

In a particularly preferred embodiment, the soy protein material used in the method of the invention is a soy protein isolate. To form the isoflavone containing soy protein isolate, a commercially available defatted soy flake material is extracted with an aqueous alkaline solution, typically a calcium hydroxide or a sodium hydroxide solution having a pH of about 6 to about 10, to form an extract containing isoflavones, protein, and other water soluble components of the soy flake material. The extract is separated from insoluble soy materials and then is treated with an acid to lower the pH of the extract to about the isoelectric point of the protein, preferably to a pH of about 4 to about 5, and most preferably to a pH of about 4.4 to about 4.6, thereby precipitating a protein curd which captures significant amounts of the isoflavones as a result of hydrogen bonding between the protein and the isoflavones as well as physical entrapment of the isoflavones in the precipitated protein. Preferably the glucoside conjugate and glucoside isoflavones are converted to aglucone isoflavones in the extract as described above to increase the amount of aglucone isoflavones captured in the protein curd. The protein curd is then separated from the extract, preferably by centrifugation, and dried to form the protein isolate.

In another preferred embodiment, the soy protein material used in the method of the invention is a soy protein concentrate. To form the isoflavone containing soy protein concentrate, a commercially available defatted soy flake material is washed with an aqueous acid having a pH at or near the isoelectric point of soy protein, preferably a pH of from 4 to 5, more preferably from 4.3 to 4.7 and most preferably a pH of 4.5. Less preferably, the commercially defatted soy flake material is washed with an alcohol, preferably an aqueous alcohol such as 80% ethanol or 80% methanol. Soy protein concentrates formed by alcohol washing are less desirable for use in the method of the present invention because the desired isoflavones are quite soluble in alcohol and significant amounts of the isoflavones are removed with the alcohol wash. The aqueous acidic or alcohol wash is subsequently separated from the protein material, leaving the soy protein concentrate. Preferably the glucoside conjugate and glucoside isoflavones are converted to aglucone isoflavones as described above to increase the amount of aglucone isoflavones in the protein concentrate.

The soy protein material may be incorporated into a food or a beverage for administration of the soy protein material. For example, a soy protein isolate or soy protein concentrate may be incorporated as an ingredient in a wide variety of foods and beverages such as meats, meat analogs, protein fortified beverages, soups, juices, cheeses, yogurts, puddings, salad dressings, ice creams, milks, and extruded snack products. Soy protein peptides are particularly suited for use in acidic beverages such as sports beverages and nutritional beverages. If desired, the soy protein can be formulated into a tablet, pill, or capsule using conventional binders and excipients for oral administration.

To prevent or inhibit stress or urge urinary incontinence, a composition containing one or more of the isoflavone compounds of Formulas 1 and/or 2 is administered on an ongoing regular basis, preferably daily, to a human in an amount effective to prevent or inhibit urinary incontinence. The isoflavone compound is administered as a pharmaceutical composition, dietary supplement composition, or as a dietary composition, depending on which route of administration is more effective and/or acceptable. The pharmaceutical composition, dietary supplement composition, or dietary composition may be formed as described above.

The particular dosage of a composition containing the isoflavone compounds administered in accordance with the present invention will depend on the route of administration, and the amount effective to prevent or inhibit stress or urge urinary incontinence in the human. Preferably the composition is administered in an amount effective to elevate the level of the isoflavone(s) in the blood of the human. An elevated level of isoflavone(s) in the blood is indicated by a blood concentration of a combination of the isoflavone(s) and its/their primary metabolites of at least 50 ng/ml(nanograms per milliliter), and more preferably a blood concentration of at least 50 ng/ml of the isoflavone(s) itself/themselves. Primary metabolites of the isoflavones of Formulas 1 and 2 in humans are equol, angolensin, O-desmethylangolensin, dihydrodaidzein, dehydroequol, 2-dehydro-O-desmethylangolensin, tetrahydrodaidzein, dihydrogenistein, and 6″-hydroxy-O-desmethylangolensin. More preferably, the blood concentration of the isoflavone compound(s) and/or its/their primary metabolites should be from about 50 ng/ml to about 10,000 ng/ml, and most preferably from about 500 ng/ml to about 5000 ng/ml to provide the desired prevention or inhibition of urinary incontinence.

A generally acceptable safe and effective daily amount of the isoflavone compounds for obtaining the desired blood concentration of the isoflavone(s) and primary metabolites to prevent or inhibit urinary incontinence is from about 2 mg/day to about 1500 mg/day, more typically from about 25 mg/day to about 1000 mg/day, and most preferably from about 50 mg/day to about 500 mg/day. Administration of a composition containing the isoflavone compounds may be adjusted to provide higher or lower daily amounts of the isoflavones and primary metabolites for humans having higher or lower body weights as appropriate.

The composition containing the isoflavone compounds may be administered in several doses per day to achieve the daily amount needed to prevent or inhibit urinary incontinence. It is preferred, however, that the daily required amount of the isoflavone compound(s) be administered in one or two doses of the isoflavone containing composition per day.

The following non-limiting formulations illustrate pharmaceutical, dietary supplement, and dietary formulations including the isoflavone compounds of Formulas 1 and 2 which may be used in accordance with the method of the present invention

### FORMULATIONS

The following Formulations 1-4 illustrate pharmaceutical and dietary supplement formulations including an isoflavone compound of Formula 1 and/or Formula 2. In the formulations, “Active ingredient” means an isoflavone compound or a mixture of isoflavone compounds of Formulas 1 and/or 2.

### Formulation 1

Gelatin Capsules

Hard gelatin capsules are prepared using the following ingredients: Active ingredient 0.1-1000 mg/capsule; Starch, NF 0-600 mg/capsule; Starch flowable powder 0-600 mg/capsule; Silicone fluid 350 centistokes 0-20 mg/capsule. The ingredients are mixed, passed through a sieve, and filled into capsules.

### Formulation 2

Tablets

Tablets are prepared using the following ingredients: Active ingredient 0.1-1000 mg/tablet; Microcrystalline cellulose 20-300 mg/tablet; Starch 0-50 mg/tablet; Magnesium stearate or stearate acid 0-15 mg/tablet; Silicon dioxide, fumed 0-400 mg/tablet; silicon dioxide, colloidal 0-1 mg/tablet, and lactose 0-100 mg/tablet. The ingredients are blended and compressed to form tablets.

### Formulation 3

Suspensions

Suspensions are prepared using the following ingredients: Active ingredient 0.1-1000 mg/5 ml; Sodium carboxymethyl cellulose 50-700 mg/5ml; Sodium benzoate 0-10 mg/5 ml; Purified water 5 ml; and flavor and color agents as needed.

### Formulation 4

Parenteral Solutions

A parenteral composition is prepared by stirring 1.5% by weight of active ingredient in 10% by volume propylene glycol and water. The solution is made isotonic with sodium chloride and sterilized.

The following Formulations 5-8 illustrate dietary compositions that may be formed using a soy protein isolate rich in several of the isoflavone compounds of Formula 1 and/ or Formula 2. The isoflavone rich soy protein isolate in the following examples typically contains between about 1 to about 8 milligrams of the isoflavone compounds per gram of soy protein.

### Formulation 5

Ready to Drink Beverage

A ready to drink beverage is formed of the following components:

| Ingredient | Percent of composition, by weight |
|---|---|
| Water | 80-85 |
| Isoflavone rich soy protein isolate | 10-15 |
| Sucrose | 5-8 |
| Cocoa | 0.1-1 |
| Vitamins/Minerals | 0.1-1 |
| Flavor | 0.1-1 |
| Cellulose gel | 0.1-0.5 |

The ready to drink beverage may be served in 8 ounce servings containing about 20 grams of isolated soy protein including about 20 to about 160 milligrams of the isoflavone compounds.

### Formulation 6

Powdered Beverage

A powdered beverage is formed of the following components:

| Ingredient | Percent of composition, by weight |
|---|---|
| Isoflavone rich soy protein isolate | 85-90 |
| Sucrose | 8-15 |
| Maltodextrin | 1-5 |
| Vitamins/Minerals | 0.5-2 |
| Aspartame | 0-0.5 |
| Flavor | 0-0.5 |

30 grams of the powdered beverage formulation may be added to water to form a serving containing about 20 grams of soy protein isolate including about 20 to about 160 milligrams of the isoflavone compounds.

### Formulation 7

Food Bar

A food bar is formed of the following components:

| Ingredients | Percent of composition, by weight |
|---|---|
| Isoflavone rich soy protein isolate | 20-30 |
| Corn syrup | 35-45 |
| Rice syrup solids | 7-14 |
| Glycerin | 1-5 |
| Cocoa | 2-7 |
| Compound coating | 15-25 |

The food bar may be served in 70 gram portions containing about 15 grams of soy protein isolate having about 15 to about 120 milligrams of the isoflavone compounds therein.

### Formulation 8

Soy Yogurt

A soy yogurt is formed of the following components:

| Ingredients | Percent of composition, by weight |
|---|---|
| Water | 65-75 |
| Isoflavone rich soy protein isolate | 5-15 |
| Sucrose | 3-8 |
| Corn starch | 1-5 |
| Dextrin | 0.3-1 |
| Cellulose gel | 1-3 |
| Culture (yogurt) | 0.01-0.1 |
| Fruit | 10-20 |
| Vitamins/Minerals | 0.05-0.3 |

The soy yogurt may be served in a 170 gram serving containing about 8 grams of soy protein isolate having about 8 to about 64 milligrams of isoflavone compounds therein.

### EXAMPLE 1

Five to fifty men are selected for clinical study. The men are diagnosed with urge incontinence. The men are divided into two groups, the first of which receives a daily dose of 100 mg of soy isoflavones in an isoflavone dietary supplement. The second group receives a placebo pill and receives no daily isoflavones (the control group). The diets of the groups are selected to include no further source of isoflavones, and no source of estrogen is administered to either of the two groups.

Prior to beginning the administration of isoflavones, the men are benchmarked as to the daily frequency of occurrence of urge incontinence. The benchmarked urge incontinence is measured again for each group after the groups have been on the isoflavone/placebo treatments for 6 months. The results are compared between members of each group and between the beginning of the study and end of the study for each member. Inhibition of urge incontinence by the isoflavones is shown by significantly greater decreased frequency of incontinence in members of the group receiving the isoflavone dietary supplement from the start of the study to the end of the treatments relative to any change in frequency of incontinence of the members of the control group throughout the study.

### EXAMPLE 2

Five to fifty women are selected for clinical study. The women are diagnosed with urge incontinence. The women are divided into two groups, the first of which receives a daily dose of 100 mg of soy isoflavones in an isoflavone dietary supplement. The second group receives a placebo pill and receives no daily isoflavones (the control group). The diets of the groups are selected to include no further source of isoflavones, and no source of estrogen is administered to either of the two groups.

Prior to beginning the administration of isoflavones, the women are benchmarked as to the daily frequency of occurrence of urge incontinence. The benchmarked urge incontinence is measured again for each group after the groups have been on the isoflavone/placebo treatments for 6 months. The results are compared between members of each group and between the beginning of the study and end of the study for each member. Inhibition of urge incontinence by the isoflavones is shown by significantly greater decreased frequency of incontinence in members of the group receiving the isoflavone dietary supplement from the start of the study to the end of the treatments relative to any change in frequency of incontinence of the members of the control group throughout the study.

### EXAMPLE 3

Five to fifty men are selected for clinical study. The men are diagnosed with stress incontinence. The men are divided into two groups, the first of which receives a daily dose of 100 mg of soy isoflavones in an isoflavone dietary supplement. The second group receives a placebo pill and receives no daily isoflavones (the control group). The diets of the groups are selected to include no further source of isoflavones, and no source of estrogen is administered to either of the two groups.

Prior to beginning the administration of isoflavones, the men are benchmarked as to the daily frequency of occurrence of stress incontinence. The benchmarked stress incontinence is measured again for each group after the groups have been on the isoflavone/placebo treatments for 6 months. The results are compared between members of each group and between the beginning of the study and end of the study for each member. Inhibition of stress incontinence by the isoflavones is shown by significantly greater decreased frequency of incontinence in members of the group receiving the isoflavone dietary supplement from the start of the study to the end of the treatments relative to any change in frequency of incontinence of the members of the control group throughout the study.

### EXAMPLE 4

Five to fifty women are selected for clinical study. The women are diagnosed with stress incontinence. The women are divided into two groups, the first of which receives a daily dose of 100 mg of soy isoflavones in an isoflavone dietary supplement. The second group receives a placebo pill and receives no daily isoflavones (the control group). The diets of the groups are selected to include no further source of isoflavones, and no source of estrogen is administered to either of the two groups.

Prior to beginning the administration of isoflavones, the women are benchmarked as to the daily frequency of occurrence of stress incontinence. The benchmarked stress incontinence is measured again for each group after the groups have been on the isoflavone/placebo treatments for 6 months. The results are compared between members of each group and between the beginning of the study and end of the study for each member. Inhibition of stress incontinence by the isoflavones is shown by significantly greater decreased frequency of incontinence in members of the group receiving the isoflavone dietary supplement from the start of the study to the end of the treatments relative to any change in frequency of incontinence of the members of the control group throughout the study.

### EXAMPLE 5

Five to fifty men are selected for clinical study. The men are diagnosed with urge incontinence. The men are divided into two groups, the first of which is provided a dietary regimen including 30 g of soy protein isolate containing 3 mg/g isoflavones daily (90 mg isoflavone/day). The second group is provided a dietary regimen including 30 g of casein (milk protein) containing no isoflavones (the control group). The diets of the groups are selected to include no further source of isoflavones, and no source of estrogen is administered to either of the two groups.

Prior to beginning the administration of dietary regimens, the men are benchmarked as to the daily frequency of occurrence of urge incontinence. The benchmarked urge incontinence is measured again for each group after the groups have been on the dietary regimens for 6 months. The results are compared between members of each group and between the beginning of the study and end of the study for each member. Inhibition of urge incontinence by the isoflavones is shown by significantly greater decreased frequency of incontinence in members of the group receiving the soy protein dietary regimen from the start of the study to the end of the dietary regimen relative to any change in frequency of incontinence of the members of the control group throughout the study.

### EXAMPLE 6

Five to fifty women are selected for clinical study. The women are diagnosed with urge incontinence. The women are divided into two groups, the first of which is provided a dietary regimen including 30 g of soy protein isolate containing 3 mg/g isoflavones daily (90 mg isoflavone/day). The second group is provided a dietary regimen including 30 g of casein (milk protein) containing no isoflavones (the control group). The diets of the groups are selected to include no further source of isoflavones, and no source of estrogen is administered to either of the two groups.

Prior to beginning the administration of dietary regimens, the women are benchmarked as to the daily frequency of occurrence of urge incontinence. The benchmarked urge incontinence is measured again for each group after the groups have been on the dietary regimens for 6 months. The results are compared between members of each group and between the beginning of the study and end of the study for each member. Inhibition of urge incontinence by the isoflavones is shown by significantly greater decreased frequency of incontinence in members of the group receiving the soy protein dietary regimen from the start of the study to the end of the dietary regimen relative to any change in frequency of incontinence of the members of the control group throughout the study.

### EXAMPLE 7

Five to fifty men are selected for clinical study. The men are diagnosed with stress incontinence. The men are divided into two groups, the first of which is provided a dietary regimen including 30 g of soy protein isolate containing 3 mg/g isoflavones daily (90 mg isoflavone/day). The second group is provided a dietary regimen including 30 g of casein (milk protein) containing no isoflavones (the control group). The diets of the groups are selected to include no further source of isoflavones, and no source of estrogen is administered to either of the two groups.

Prior to beginning the administration of dietary regimens, the men are benchmarked as to the daily frequency of occurrence of stress incontinence. The benchmarked stress incontinence is measured again for each group after the groups have been on the dietary regimens for 6 months. The results are compared between members of each group and between the beginning of the study and end of the study for each member. Inhibition of stress incontinence by the isoflavones is shown by significantly greater decreased frequency of incontinence in members of the group receiving the soy protein dietary regimen from the start of the study to the end of the dietary regimen relative to any change in frequency of incontinence of the members of the control group throughout the study.

### EXAMPLE 8

Five to fifty women are selected for clinical study. The women are diagnosed with stress incontinence. The women are divided into two groups, the first of which is provided a dietary regimen including 30 g of soy protein isolate containing 3 mg/g isoflavones daily (90 mg isoflavone/day). The second group is provided a dietary regimen including 30 g of casein (milk protein) containing no isoflavones (the control group). The diets of the groups are selected to include no further source of isoflavones, and no source of estrogen is administered to either of the two groups.

Prior to beginning the administration of dietary regimens, the women are benchmarked as to the daily frequency of occurrence of stress incontinence. The benchmarked stress incontinence is measured again for each group after the groups have been on the dietary regimens for 6 months. The results are compared between members of each group and between the beginning of the study and end of the study for each member. Inhibition of stress incontinence by the isoflavones is shown by significantly greater decreased frequency of incontinence in members of the group receiving the soy protein dietary regimen from the start of the study to the end of the dietary regimen relative to any change in frequency of incontinence of the members of the control group throughout the study.

The present invention is not limited to the above embodiments and can be variously modified. The above description of preferred embodiments is intended only to acquaint others skilled in the art with the invention, its principles and its practical application so that others skilled in the art may adapt and apply the invention in its numerous forms, as may be best suited to the requirements of a particular use. With reference to the use of the word(s) “comprise” or “comprises” or “comprising” in this entire specification (including the claims below), it is noted that unless the context requires otherwise, those words are used on the basis and clear understanding that they are to be interpreted inclusively, rather than exclusively, and that it is intended each of those words to be so interpreted in construing this entire specification.

1. A method for preventing or inhibiting stress or urge urinary incontinence comprising:
administering to a human a composition containing at least one isoflavone selected from the group consisting of genistein, daidzein, glycitein, biochanin A, formononetin, their naturally occurring glycosides, their naturally occurring glycoside conjugates, or mixture thereof in an amount which is effective to prevent or inhibit stress or urge urinary incontinence in said human.

2. The method of claim 1 wherein said composition is administered to said human in an amount effective to elevate the level of said isoflavone in said human, where an elevated level of said isoflavone in said human is indicated by a blood concentration of at least 50 ng/ml of said isoflavone and primary metabolites of said isoflavone.

3. The method of claim 1, wherein the isoflavone is administered in an amount of from about 2 to about 1500 milligrams per day.

4. The method of claim 1, wherein the isoflavone is administered in an amount of from about 25 to about 1000 milligrams per day.

5. The method of claim 1, wherein the isoflavone is administered in an amount of from about 50 to about 500 milligrams per day.

6. The method of claim 1, wherein said isoflavone in said composition is genistein, daidzein, or a mixture thereof.

7. The method of claim 1 wherein at least one of the isoflavones in said composition is derived from soy or clover.

8. The method of claim 1 wherein said composition is a pharmaceutical or a dietary supplement composition containing an excipient and said isoflavone.

9. The method of claim 8 wherein said pharmaceutical or dietary supplement composition is a pill or a capsule.

10. The method of claim 8 wherein said pharmaceutical or dietary supplement composition is a suspension or a solution.

11. The method of claim 10 wherein said pharmaceutical composition is capable of being administered subcutaneously.

12. The method of claim 10 wherein said pharmaceutical composition is capable of being administered transdermally.

13. The method of claim 8 wherein said pharmaceutical or dietary supplement composition is capable of being administered orally.

14. The method of claim 1 wherein said composition is a dietary composition containing a food ingredient and said isoflavone compound.

15. The method of claim 14 wherein said food ingredient is a soy protein material.
